SEQUENCE LISTING (41 sequences)

SEQ ID NO: 1 - protein, 359 aa, Homo sapiens
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKEYNLV

SEQ ID NO: 2 - protein, 359 aa, Homo sapiens
MTLESMMACCLSDEVKESKRINAEIEKQLRRDKRDARRELKLLLLGTGESGKSTFIKQMRIIHGAGYSEEDKRGFTKLVYQNIFTAMQAMIRAMETLKILYKYEQNKANALLIREVDVEKVTTFEHQYVSAIKTLWEDPGIQECYDRRREYQLSDSAKYYLTDVDRIATLGYLPTQQDVLRVRVPTTGIIEYPFDLENIIFRMVDVGGQRSERRKWIHCFENVTSIMFLVALSEYDQVLVESDNENRMEESKALFRTIITYPWFQNSSVILFLNKKDLLEDKILYSHLVDYFPEFDGPQRDAQAAREFILKMFVDLNPDSDKIIYSHFTCATDTENIRFVFAAVKDTILQLNLKEYNLV

SEQ ID NO: 3 - protein, 9 aa, synthetic construct
IMDQVPFSV

SEQ ID NO: 4 - protein, 9 aa, synthetic construct
YLEPGPVTA

SEQ ID NO: 5 - protein, 9 aa, synthetic construct
ALLAVGATK

SEQ ID NO: 6 - protein, 9 aa, synthetic construct
LIYRRRLMK

SEQ ID NO: 7 - protein, 12 aa, synthetic construct
DAEKSDICTDEY

SEQ ID NO: 8 - protein, 11 aa, synthetic construct
SSDYVIPIGTY

SEQ ID NO: 9 - protein, 9 aa, synthetic construct
YMDGTMSQV

SEQ ID NO: 10 - protein, 9 aa, synthetic construct
EADPTGHSY

SEQ ID NO: 11 - protein, 9 aa, synthetic construct
SLFRAVITK

SEQ ID NO: 12 - protein, 9 aa, synthetic construct
EVDPIGHLY

SEQ ID NO: 13 - protein, 9 aa, synthetic construct
GLYDGMEHL

SEQ ID NO: 14 - protein, 10 aa, synthetic construct
ASGPGGGAPR

SEQ ID NO: 15 - protein, 19 aa, synthetic construct
IFRMVDVGGLRSERRKWIH

SEQ ID NO: 16 - protein, 19 aa, synthetic construct
IFRMVDVGGPRSERRKWIH

SEQ ID NO: 17 - protein, 16 aa, synthetic construct
WNRQLYPEWTEAQRLD

SEQ ID NO: 18 - protein, 16 aa, synthetic construct
AQNILLSNAPLGPQFP

SEQ ID NO: 19 - protein, 21 aa, synthetic construct [sequence omitted]

SEQ ID NO: 20 - protein, 23 aa, synthetic construct
RNGYRALMDKSLHVGTQCALTRR

SEQ ID NO: 21 - protein, 14 aa, synthetic construct
LLKYRAREPVTKAE

SEQ ID NO: 22 - protein, 15 aa, synthetic construct
TSYVKVLHHMVKISG

SEQ ID NO: 23 - protein, 30 aa, synthetic construct
GARGPESRLLEFYLAMPFATPMEAELARRS

SEQ ID NO: 24 - protein, 31 aa, synthetic construct
FDLQSVIFRMVDVGGQRSERRKWIHCFENVT

SEQ ID NO: 25 - protein, 31 aa, synthetic construct
FDLENIIFRMVDVGGQRSERRKWIHCFENVT

SEQ ID NO: 26 - protein, 9 aa, synthetic construct
SLLQHLIGL

SEQ ID NO: 27 - protein, 9 aa, synthetic construct
ELFSYLIEK

SEQ ID NO: 28 - protein, 10 aa, synthetic construct
RPRRWKLQVL

SEQ ID NO: 29 - protein, 9 aa, synthetic construct
KVKRKKNVL

SEQ ID NO: 30 - protein, 19 aa, synthetic construct
TSPRRLVELAGQSLLKDEA

SEQ ID NO: 31 - protein, 19 aa, synthetic construct
DELFSYLIEKVKRKKNVLR

SEQ ID NO: 32 - protein, 31 aa, synthetic construct
YPFDLENIIFRMVDVGGLRSERRKWIHCFEN

SEQ ID NO: 33 - protein, 31 aa, synthetic construct
YPFDLQSVIFRMVDVGGLRSERRKWIHCFEN

SEQ ID NO: 34 - protein, 31 aa, synthetic construct
YPFDLQSVIFRMVDVGGPRSERRKWIHCFEN

SEQ ID NO: 35 - protein, 25 aa, synthetic construct
FDLENIIFRMVDVGGLRSERRKWIH

SEQ ID NO: 36 - protein, 25 aa, synthetic construct
FDLQSVIFRMVDVGGLRSERRKWIH

SEQ ID NO: 37 - protein, 25 aa, synthetic construct
FDLQSVIFRMVDVGGPRSERRKWIH

SEQ ID NO: 38 - protein, 9 aa, synthetic construct
RMVDVGGLR

SEQ ID NO: 39 - protein, 10 aa, synthetic construct
FRMVDVGGLR

SEQ ID NO: 40 - protein, 9 aa, synthetic construct
RMVDVGGPR

SEQ ID NO: 41 - protein, 9 aa, synthetic construct
GPRSERRKW

(19) United States
(12) Patent Application Publication
(10) Pub. No.: US 2026/0053905 A1
(43) Pub. Date: Feb. 26, 2026

(54) UVEAL MELANOMA VACCINE

(71) Applicant: University of Virginia Patent Foundation, Charlottesville, VA (US)

(72) Inventor: Craig Slingluff, Charlottesville, VA (US)

(21) Appl. No.: 19/307,940

(22) Filed: Aug. 22, 2025

Related U.S. Application Data
(60) Provisional application No. 63/685,987, filed on Aug. 22, 2024.

(51) Int. Cl. A61K 39/00

(52) U.S. Cl. CPC A61K 39/001186; A61K 39/0011; A61K 39/001156; A61K 39/001188; A61K 39/001189; A61K 39/001191; A61K 39/001192; A61K 2039/54; A61K 2039/545

(57) ABSTRACT

Disclosed are multi-peptide compositions and/or methods of use of the multi-peptide compositions for patients with resected (or definitively treated) high-risk uveal melanoma. Pharmaceutically acceptable embodiments administered to patients may raise an immune system response against uveal melanoma cells, leading to a decrease in uveal melanoma cell number. Further embodiments may take the form of an adjuvant therapy or a neoadjuvant therapy, as monotherapy or in combination with other therapies.

### CROSS-REFERENCE TO RELATED APPLICATIONS

The instant application claims the benefit of priority to U.S. Provisional Patent Application No. 63/685,987 filed on 22 Aug. 2024, the entire contents of which are incorporated herein by reference.

### SEQUENCE LISTING

The instant application contains a Sequence Listing which has been submitted electronically in ST.26.xml format and is hereby incorporated by reference in its entirety. Said ST.26 copy, created on 22 Aug. 2025 is named “20250822UVA019US2SEQ” and is 40,960 bytes in size.

### BACKGROUND

1. Field of the discovery. Embodiments of the disclosure presented herein relate generally to the field of uveal melanoma and more specifically to a multi-peptide composition and/or method of use of the multi-peptide compositions for patients with resected (or definitively treated) high-risk uveal melanoma. Pharmaceutically acceptable embodiments administered to patients may raise an immune system response against uveal melanoma cells, leading to a decrease in uveal melanoma cell number. Further embodiments may take the form of an adjuvant therapy.

2. Background information. Uveal melanoma (UM) is the most common malignancy of the eye, representing 4-5% of all melanomas. Like cutaneous melanomas, it arises from melanocytes (originally cells of the neural crest), but its clinical behavior differs from that of cutaneous melanomas. It occurs in about 3,000 Americans annually. Among those with stage IV disease, less than 10% survive more than 5 years. Primary uveal melanomas at highest risk for metastasis can be identified by a gene expression profiling test (DecisionDx-UM™). Among patients with high-risk (Class 2) tumors, more than half develop metastases within 5 years, compared to only 13% for patients with low-risk (Class 1) tumors. The most common metastatic site is the liver. There are no curative therapies for metastatic uveal melanoma. Thus, earlier intervention for high-risk uveal melanomas is needed.

Immune checkpoint blockade therapy (CBT) has transformed management of cutaneous melanomas, but clinical responses to CBT have been very low for uveal melanomas. Tumor mutation burden (TMB) is very low for the vast majority of uveal melanomas, with a median of 13 single nucleotide variations (SNVs) per genome (range 5-33) for primary uveal melanomas and 16 SNVs per genome (range 5-33) for metastatic uveal melanomas. TMB has been associated with clinical response to CBT. Consistent with that, a meta-analysis of outcomes of 1414 patients with UM revealed an overall objective clinical response rate of only 3.4% (95% CI 1.8-5.1%) for single agent CBI and 12.4% (95% CI: 8-16.9%) for blockade of PD-1 and CTLA4, with median overall survival of just 11 months overall.

The only approval therapy for metastatic uveal melanoma is tebentafusp, which improves 5-year survival from <10% to ˜15%. Tebentafusp is a bispecific molecule for CD3 and the T cell receptor for gp100₂₈₀₋₂₈₈ peptide (YLEPGPVTA) bound to HLA-A*0201 molecules on tumor cells. It also enhanced overall survival for HLA-A*0201+ patients vs investigator choice of pembrolizumab, ipilimumab or dacarbazine⁶ and other CBT. Thus, the only effective therapy for advanced uveal melanoma is an immune therapy targeting T cells to a melanocytic differentiation antigen (gp100) peptide presented on HLA-A*0201 molecules. Since this only antigen-directed therapy approved in UM is restricted by HLA-A*0201, which is expressed by about 45% of the UM population, there is no approved therapy for the majority of UM patients. Even for patients who Express HLA-A*0201, most patients still die of their disease. Thus, there is an urgent need for new effective antigen-directed therapies for uveal melanoma that may be broadly applicable across a wide range of HLA types.

### SUMMARY

Disclosed are multi-peptide compositions and/or methods of use of the multi-peptide compositions for patients with resected (or definitively treated) high-risk uveal melanoma. Pharmaceutically acceptable embodiments administered to patients may raise an immune system response against uveal melanoma cells, leading to a decrease in uveal melanoma cell number. Further embodiments may take the form of an adjuvant therapy. In certain other embodiments the peptides may be applied as part of a neoadjuvant therapy.

Disclosed herein is a multi-peptide vaccine composition designed for the treatment of uveal melanoma. The composition includes peptides selected from key melanoma-associated source proteins, specifically gp100, tyrosinase, MelanA/MART-1, MAGE-A1, MAGE-A3, MAGE-A10, PRAME, and NY-ESO-1. These peptides are chosen for their ability to bind Class I major histocompatibility complex (MHC) molecules, thereby stimulating cytotoxic T cell responses.

Additionally, the composition contains peptides from gp100, tyrosinase, MelanA/MART-1, MAGE-A1, MAGE-A3, MAGE-A6, and PRAME selected for binding to Class II MHC molecules to activate helper T cell responses. The vaccine further incorporates at least one peptide comprising a mutated sequence from GNAQ or GNA11, with the mutation occurring at residue 209 and selected from Q209L or Q209P, targeting common driver mutations in uveal melanoma.

An example embodiment consists of twenty-six peptides: sixteen Class I MHC-binding peptides, eight Class II MHC-binding peptides, and two mutated peptides (Q209L and Q209P). Specific sequences for these peptides are provided, including:

Class I MHC-binding peptides:

(SEQ ID NO 3)
IMDQVPFSV
(SEQ ID NO 4)
YLEPGPVTA
(SEQ ID NO 5)
ALLAVGATK
(SEQ ID NO 6)
LIYRRRLMK
(SEQ ID NO 7)
DAEKSDICTDEY
(SEQ ID NO 8)
SSDYVIPIGTY
(SEQ ID NO 9)
YMDGTMSQV
(SEQ ID NO 10)
EADPTGHSY
(SEQ ID NO 11)
SLFRAVITK
(SEQ ID NO 12)
EVDPIGHLY
(SEQ ID NO 13)
GLYDGMEHL
(SEQ ID NO 14)
ASGPGGGAPR
(SEQ ID NO 26)
SLLQHLIGL
(SEQ ID NO 27)
ELFSYLIEK
(SEQ ID NO 28)
RPRRWKLQVL
(SEQ ID NO 29)
KVKRKKNVL

Class II MHC-binding peptides:

(SEQ ID NO 17)
WNRQLYPEWTEAQRLD
(SEQ ID NO 18)
AQNILLSNAPLGPQFP
(SEQ ID NO 19)
FLLHHAFVDSIFEQWLQRHRP
(SEQ ID NO 20)
RNGYRALMDKSLHVGTQCALTRR
(SEQ ID NO 21)
LLKYRAREPVTKAE
(SEQ ID NO 22)
TSYVKVLHHMVKISG
(SEQ ID NO 30)
TSPRRLVELAGQSLLKDEA
(SEQ ID NO 31)
DELFSYLIEKVKRKKNVLR

Mutated peptides:

(Q209L, SEQ ID NO 15)
IFRMVDVGGLRSERRKWIH
(Q209P, SEQ ID NO 16)
IFRMVDVGGPRSERRKWIH

The vaccine is formulated as a pharmaceutically acceptable preparation, such as a water-in-oil emulsion with incomplete Freund's adjuvant and is suitable for intradermal or subcutaneous administration. Formulations for alternative routes of introduction such as intramuscular, optical, nasal, respirator, etc. are possible with the usage of standard carriers. Each peptide is synthesized under GMP conditions and provided in lyophilized single-use vials, with optional carriers including water, saline, lactated Ringer's solution, dextrose solution, or human albumin. Other formulations and forms are possible such as are known, for example, pre-made solution or combinations of one or more peptides.

A kit for administration may be provided, containing the multi-peptide composition, buffer or media for resuspension, instructions for use, and packaging materials for storage and transport. Methods of use include administration to a human, with dosing regimens that may involve pre-treatment with cyclophosphamide, multiple injection sites, and booster doses at three-week intervals for up to two years in the absence of disease progression.

Additional embodiments include shorter mutated peptides for GNAQ and GNA11, such as:

(SEQ ID NO 38)
RMVDVGGLR
(SEQ ID NO 39)
FRMVDVGGLR
(SEQ ID NO 40)
RMVDVGGPR
(SEQ ID NO 41)
GPRSERRKW

These shorter peptides are designed to stimulate CD8 T cell responses directly across multiple HLA molecules. The composition is specifically tailored to address the heterogeneity of uveal melanoma and improve therapeutic outcomes through multi-antigen and neoantigen targeting.

In embodiments, longer peptides targeting CD4 T cell activation may be combined with shorter peptides targeting CD8 T cell activation.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages objects and embodiments are expressly included within the scope of the present disclosure. The publications and other materials used herein to illuminate the background of the disclosure, and in particular cases, to provide additional details respecting the practice, are incorporated by reference.

### BRIEF DESCRIPTION OF THE DRAWINGS

The accompanying drawings, which are incorporated into and form a part of the specification, illustrate several embodiments of the present disclosure; and, together with the description, serve to explain the principles of the disclosure. The drawings are only for the purpose of illustrating an embodiment of the disclosure and are not to be construed as limiting the disclosure. Further objects, features and advantages of the disclosure will become apparent from the following detailed description taken in conjunction with the accompanying figures showing illustrative embodiments of the disclosure, in which:

FIG. 1. Presents a protocol schema for the administration of a UM vaccine and/or adjuvant treatment.

### DETAILED DESCRIPTION

Disclosed is a multi-peptide composition and/or method of use of the composition for patients with resected (or definitively treated) high-risk uveal melanoma. Pharmaceutically acceptable embodiments administered to patients may raise an immune system response against uveal melanoma cells, leading to a decrease in uveal melanoma cell number. Further embodiments may take the form of an adjuvant therapy.

Current melanoma vaccines stimulate CD4 and CD8 T cells across a wide range of HLA alleles. The targets of these vaccines include gp100 and other melanocytic differentiation proteins, as well as cancer-testis antigens, with high expression also in uveal melanomas. Embodiments of the disclosure use embodiments of the herein disclosed UM vaccines as adjuvant or neoadjuvant therapy for high-risk patients with uveal melanoma. In addition to the current antigens, embodiments of the disclosure add peptides from the most common shared driver mutations in uveal melanoma (GNAQ and GNA11), for which multiple Class I MHC-restricted epitopes have been predicted in silico. These shared mutated neoantigens offer promise for inducing immune responses across almost all uveal melanoma patients. In certain embodiments, additional antigens are mutated GNAQ and mutated GNA11. In certain embodiments the multi-peptide composition contains twenty (20) peptides overall: twelve (12) melanoma peptides (12MP) and six (6) melanoma helper peptides (6MHP) with two (2) mutated peptides GNAQ and GNA11. This combination is referred to as “mEyeVax20” (i.e., 12MP+6MHP+GNAQ/GNA11 peptides). In certain other embodiments additional peptides are sourced from one or more PRAME proteins.

While various embodiments of the present disclosure are described herein, it will be understood by those skilled in the art that such embodiments are provided by way of example only. It will be understood by those skilled in the art that numerous modifications and changes to, and variations and equivalent substitutions of, the embodiments described herein can be made without departing from the scope of the disclosure. It is understood that various alternatives to the embodiments described herein may be employed in practicing the disclosure, and modifications may be made to adapt a particular structure or material to the teachings of the disclosure. It is also understood that every embodiment of the disclosure may optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether the specific exclusion is recited in the specification.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

Unless otherwise defined, all technical and scientific terms used herein have the same meaning as commonly understood by one of ordinary skill in the art to which this disclosure belongs.

Where a range of values is provided, it is understood that each intervening value between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding both of those included limits are also included in the disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The term “exemplary” as used herein means “serving as an example, instance or illustration”. Any embodiment or feature characterized herein as “exemplary” is not necessarily to be construed as preferred or advantageous over other embodiments or features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

As used herein the term “vaccine” is used as ordinarily understood (e.g., a preparation used to stimulate an immune response against one or more diseases) and may also contain a secondary descriptor describing the antigenic component eliciting the desired immune response along with the associated ingredients tailored to the desired effect. It is further understood that a vaccine may be all or part of a “pharmaceutical preparation” as herein defined and may incorporate a combination of natural and synthetic components. In certain instances, it is recognized in the art that bringing together two natural components, a viral piece and an adjuvant, give rise to a composition with properties greater than the sum of its parts. (i.e., conferring a greater immune response, providing stabilization of antigens, etc.) It is those enhanced, non-naturally occurring properties to which the term “vaccine” further contemplates.

As used herein, the term “polyvalent vaccine” refers to multiple antigen types that may derive from a single or multiple disease vectors whereas a “combination vaccine” refers to a vaccine with multiple antigens targeting at least two or more diseases. Thus, a “polyvalent vaccine” may have multiple antigens but only provide immunity to a single disease whereas a “combination vaccine” may contain a single type of antigen from two or more disease vectors and give rise to an immunity for those two or more diseases. Also, as used herein, the term “patient” reflects a human or animal subject to whom a medical treatment is provided either for modification of a disease state or as a prophylactic preventative of disease.

The term “antigens” and derivatives are defined as the components typically derived from the structure of disease-causing organisms, which are recognized as “foreign” by the immune system and trigger a protective immune response to the vaccine. Importantly, the antigen component does not have to be made by the disease-causing organism in order to be used. For example, viral proteins or protein precursors may be manufactured and purified using bacterial, fungal, cell free, or other vector-based systems in bulk and then purified for use in a vaccine serum or other pharmaceutical preparation. In certain embodiments, antigens may include one or more polypeptides.

Vaccines may be administered through multiple routes at least including: intramuscular injection, subcutaneous injection, intradermal injection, orally, or through intranasal sprays. It is understood that each route of administration may require its own mix of antigens and associated ingredients.

Pharmaceutical preparations or compositions described or used herein may further comprise coloring or stabilizing agents, osmotic agents, antibacterial agents, or any other substances if such substances do not interfere with the function of the composition. The pharmaceutical compositions of the instant disclosure, can, for example, be formulated as a solution, suspension, or emulsion in association with a pharmaceutically acceptable parenteral vehicle. Examples of such vehicles are water, saline, Ringer's solution, dextrose solution, and 5% human albumen. Liposomes may also be used. The vehicle may contain additives that maintain isotonicity (e.g., sodium chloride or mannitol) and chemical stability (e.g., buffers and preservatives). It should be appreciated that endotoxin contamination should be kept at a safe level, for example, less than 0.5 ng mg-1 protein. Moreover, for human administration, preparations should meet sterility, pyrogenicity, general safety and purity standards as required by the United States Food and Drug Administration Office of Biological Standards. The formulations may be sterilized by commonly used techniques such as filtration.

The phrase “pharmaceutically acceptable” refers to substances and compositions which do not produce an adverse, allergic, or otherwise untoward reaction when administered to an animal, or a human, as appropriate. A substance which caused or produced any of these adverse effects would be classified as “biologically harmful” within the scope of the present disclosure. Pharmaceutically acceptable substances and compositions include, but are not limited to solvents, dispersion media, coatings, antibacterial and antifungal agents, isotonic and absorption delaying agents. Except where incompatible with the disclosure the use of any conventional ingredient is contemplated. Furthermore, supplementary active ingredients which serve some other pharmacologically expedient purpose can also be incorporated into the instant compositions without departing from the broader scope of the instant disclosure.

As used herein the term “adjuvant therapy” is used as commonly understood in the field of oncology. In typical use, adjuvant therapy is a cancer treatment that is given after primary treatments, such as surgery. Adjuvant therapy targets cancer cells (e.g., UM cells) that the primary cancer treatment did not destroy. Types of adjuvant therapy can include: chemotherapy, hormone therapy, radiation therapy, immunotherapy, or target therapy. Adjuvant therapies may be administered alone or in combination with each other according to those criteria known to those of skill in the art. Additional embodiments may be used as part of a “neoadjuvant therapy” protocol. As used herein, the term “neoadjuvant therapy” is used as commonly understood in the field of oncology. In typical use, a neoadjuvant therapy is provided before a main treatment (e.g., a surgery). Neoadjuvant therapy may sometimes be referred to by those of ordinary skill in the art as “pre-adjuvant therapy.” Embodiments may be administered in one or both settings or may be altered with a first embodiment applied as a neoadjuvant and a second embodiment applied as an adjuvant therapy.

The effective dose and method of administration of a particular embodiment of the instant disclosure may vary based on the individual patient and stage of any present diseases (e.g., breast cancer, HIV, other co-morbidities), as well as other factors known to those of skill in the art. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

Toxicity and safe dosage levels may be determined through the determination of dose-limiting toxicities (DLTs) and the overall DLT-rate (e.g., such as in the context of a clinical trial). In certain embodiments compositions with a DLT rate less than 25% are considered safe.

Effectiveness of embodiments disclosed herein may further be evaluated through cohort studies and examination of the recurrence-free survival (RFS) rate at chosen time intervals.

The exact dosage is chosen by an individual physician in view of a patient to be treated. Dosage and administration are adjusted to provide sufficient levels of embodiments of the instant disclosure to maintain the desired effect (e.g., inducement of an immune response against uveal melanoma). Additional factors that may be taken into account include the severity of any disease state, age, weight, and gender of the patient; diet, time and frequency of the administration, drug combination(s), reaction sensitivities, and tolerance/response to therapy. Short acting pharmaceutical compositions are administered daily whereas long-acting pharmaceutical compositions are administered every 2, 3 to 4 days, every week, or once every two weeks or more. Depending on half-life and clearance rate of the particular formulation, the pharmaceutical compositions of the instant disclosure may be administered once, twice, three, four, five, six, seven, eight, nine, ten or more times per day.

Normal dosage amounts may vary from approximately 1 to 100,000 micrograms, up to a total dose of about 10 grams, depending upon the route of administration. Desirable dosages include 250 ug, 500 μg, 1 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1 g, 1.1 g, 1.2 g, 1.3 g, 1.4 g, 1.5 g, 1.6 g, 1.7 g, 1.8 g, 1.9 g, 2 g, 3 g, 4 g, 5 g, 6 g, 7 g, 8 g, 9 g, and 10 g.

More specifically, the dosage of peptide agents described herein is one that provides sufficient peptide agent to attain a desirable effect, including stimulation of the immune system to induce a treatment effect. Accordingly, the dose of the peptide agent preferably produces a tissue or blood concentration of both about 1 to 800 μM. Preferable doses produces a tissue or blood concentration of greater than about 10 μM to about 500 μM. Preferable doses are, for example, the amount of peptide required to achieve a tissue or blood concentration or both of 10 μM, 15 μM, 20 μM, 25 μM, 30 μM, 35 μM, 40 μM, 45 μM, 50 μM, 55 μM, 60 M, 65 μM, 70 μM, 75 μM, 80 μM, 85 μM, 90 μM, 95 μM, 100 μM, 110 μM, 120 μM, 130 μM, 140 μM, 150 μM, 160 μM, 170 μM, 180 μM, 190 μM, 200 μM, 220 μM, 240 μM, 250 μM, 260 μM, 280 μM, 300 μM, 320 μM, 340 μM, 360 μM, 380 μM, 400 μM, 420 μM, 440 μM, 460 μM, 480 μM, and 500 μM. Although doses that produce a tissue concentration greater than 800 μM are not necessarily preferred, they are envisioned and can be used with some embodiments of the present disclosure. A constant infusion of embodiments of the disclosure can be provided to maintain a stable concentration of the therapeutic agents.

The pharmacologically active compounds of this invention can be processed in accordance with conventional pharmaceutical practices to produce medicinal agents for administration to patients (e.g., mammals including humans). The peptides with, or without, modification can be incorporated into a pharmaceutical composition. Further, the manufacture of pharmaceuticals or therapeutic agents that deliver the peptides or a nucleic acid sequence encoding a peptide by several routes is an embodiment.

As used herein the term “sequence” explicitly contemplates DNA, cDNA, RNA and resulting peptide chains encoded thereby in both sense and antisense directions. To know one is to know the others via the standard rules of complementarity and codon encoding as exemplified in standardized DNA, RNA, and amino acid codon tables.

A “peptide” in the context of the present disclosure is to be understood as meaning a polymer composed of amino acids, preferably the 20 proteinogenic L-amino acids, preferably of linear structure, which has up to 100 amino acids which are linked to one another via peptide bonds. According to the disclosure, the peptides of the disclosure have an amino acid sequence of 4 to 50 amino acids. In the context of this disclosure, the amino acids are given in a one-letter code, where, for example, C stands for cysteine, R for arginine, A for alanine and L for leucine. It is further understood that unless otherwise indicated, the amino acids in an amino acid sequence disclosed herein are linked via peptide bonds and, unless otherwise indicated, the sequence is listed in N- to C-terminal orientation.

Peptides can be chemically synthesized in various embodiments and/or recombinantly produced using protein design. Short peptides can easily be prepared synthetically, for example via solid phase synthesis. Longer peptides and polypeptides, on the other hand, are often produced recombinantly in A host organism.

Typical acidic or negatively charged amino acids (depending on pH) are D and E.

The positively charged or basic amino acids (depending on the pH value) typically include R, K and H.

Amino acids such as G, A, C, I, L, M, F, V, P, S, T, W, Y, N and Q are typically uncharged, i.e. neutral, amino acids.

When reference is made herein to an “any” amino acid, what is commonly meant is one of the 20 naturally occurring proteinogenic amino acids, i.e. one of glycine (G), alanine (A), valine (V), leucine (L), isoleucine (I), Phenylalanine (F), Serine(S), Threonine (T), Proline (P), Methionine (M), Cysteine (C), Histidine (H), Lysine (K), Arginine (R), Glutamine (Q), asparagine (N), aspartic acid (D), glutamic acid (E), tyrosine (Y) and tryptophan (W). Unless otherwise stated, the amino acids are typically L-amino acids. In alternative embodiments, the peptide can also consist of D-amino acids, although it may be preferred that D- and L-amino acids do not occur at the same time within the peptides described herein. In various embodiments, any such amino acid includes all the aforementioned amino acids with the exception of proline, or in some embodiments also with the exception of proline and glycine.

The identity of nucleic acid or amino acid sequences is determined by sequence comparison. This sequence comparison is based on the BLAST algorithm established and commonly used in the art (e.g., Altschul et al. (1990) Basic local alignment search tool, J. Mol. Biol., 215:403-410, and Altschul et al. (1997) Gapped BLAST and PSI-BLAST: a new generation of protein database search programs, Nucleic Acids Res., 25:3389-3402) and basically happens by similar sequences of nucleotides or amino acids in the nucleic acid or amino acid sequences be assigned. A tabular assignment of the relevant positions is called alignment. Another algorithm available in the art is the FASTA algorithm. Sequence comparisons (alignments), especially multiple sequence comparisons, are created using computer programs. For example, the Clustal series (see e.g. Chenna et al. (2003) Multiple sequence alignment with the Clustal series of programs, Nucleic Acid Res., 31:3497-3500), T-Coffee (see e.g. Notredame et al. (2000) T-Coffee: A novel method for multiple sequence alignments, J. Mol. Biol., 302:205-217) or programs based on these programs or algorithms. Sequence comparisons (alignments) are also possible using the computer program Vector NTI® Suite 10.3 (Invitrogen Corporation, 1600 Faraday Avenue, Carlsbad, California, USA) with the specified standard parameters, whose AlignX module for sequence comparisons is based on ClustalW, or Clone Manager 10 (Use of the BLOSUM 62 scoring matrix for sequence alignment at the amino acid level). Unless otherwise stated, sequence identity reported herein is determined using the BLAST algorithm.

Such a comparison also allows a statement to be made about the similarity of the compared sequences to one another. It is usually given as percent identity, i.e. the proportion of identical nucleotides or amino acid residues in the same positions or in positions corresponding to one another in an alignment. The broader concept of homology includes conserved amino acid exchanges in amino acid sequences, i.e. amino acids with similar chemical activity, since these usually exert similar chemical activities within the protein. Therefore, the similarity of the compared sequences can also be stated as percent homology or percent similarity. Identity and/or homology information can be made for entire polypeptides or genes or just for individual regions. Homologous or identical regions of different nucleic acid or amino acid sequences are therefore defined by matches in the sequences. Such areas often have identical functions. They can be small and contain only a few nucleotides or amino acids. Such small areas often perform essential functions for the overall activity of the protein. It can therefore make sense to relate sequence matches only to individual, possibly small areas. Unless otherwise stated, identity or homology information in the present application refers to the total length of the nucleic acid or amino acid sequence specified in each case.

The peptide or protein concentration can be determined using known methods, for example the BCA method (bicinchoninic acid; 2,2′-biquinolyl-4,4′-dicarboxylic acid) or the biuret method (Gornall et al., J. Biol. Chem., 1948, 177:751-766). Those skilled in the art of peptide and protein technology will be aware of a variety of suitable methods for determining peptide or protein concentration that can be used within the scope of this disclosure.

Peptides according to the disclosure can have amino acid changes, in particular amino acid substitutions, insertions, or deletions. Such peptides are further developed, for example, through targeted genetic modification, i.e. through mutagenesis processes, and optimized for specific purposes or with regard to special properties (e.g., in terms of their stability, binding, etc.).

For example, targeted mutations such as substitutions, insertions or deletions can be introduced into the known molecules in order to change certain properties, for example. For this purpose, in particular the surface charges and/or the isoelectric point of the molecules and thereby their interactions with a surface can be changed. For example, the net charge of the peptides can be changed in order to influence substrate binding. Alternatively or additionally, one or more corresponding mutations can, for example, increase the stability or adsorption of the peptide. Advantageous properties of individual mutations, e.g. individual substitutions, can complement each other.

The term “conservative amino acid substitution” means the exchange (substitution) of an amino acid residue for another amino acid residue, whereby this exchange does not lead to a change in polarity or charge at the position of the exchanged amino acid, e.g. the exchange of a non-polar amino acid residue for another non-polar amino acid residue. Conservative amino acid substitutions within the scope of the disclosure include, for example: G=A=S, I=V=L=M, D=E, N=Q, K=R, Y=F, S=T, G=A=I=V=L=M=Y=F=W=P=S=T. In a similar sense, the term “conservative substitution” may also refer to one or more substitutions that do not impact an active site or other functioning of a protein encoded by a peptide.

In preferred embodiments, the peptide according to the disclosure can also be modified. Preferred modifications can be, for example, coupling the peptide with certain other molecules or chemical groups, for example organic (macro) molecules, for example via a covalent bond or a linker/spacer via a suitable amino acid of the chain and/or N- and/or C-terminal.

All of the aforementioned features and embodiments can be implemented individually or in any combination.

Furthermore, the peptide according to the disclosure can also be at least one subunit (module) of a larger peptide or polypeptide, where the polypeptide can comprise a multimer of the sequences described herein, for example 1 to 30 repeats, more preferably 2 to 15 repeats, particularly preferably 2 to 10 repeats, e.g. 2, 3, 4, 5 or 6 repeats of the peptide. The polypeptide may include or consist of such multimers. The term “polypeptide” in this context refers in particular to those peptides that comprise 100 or more amino acids. The term “larger peptides” preferably refers to peptides with at least 40 amino acids, unless otherwise described.

In various embodiments, the peptide is a peptide or polypeptide (multimer) comprising two or more of the peptides as described herein. In various embodiments, the two or more peptides can be connected to one another by at least one spacer, preferably the at least one spacer comprises or consists of 1 to 10 amino acid residues, in particular 2, 3 or 4 amino acid residues, preferably selected from the group consisting of G, P, I, A and S or combinations thereof, in particular GPI or GAS. In such embodiments, the individual peptides are optionally connected linearly to one another via peptide bonds, possibly also via a spacer.

The peptides described herein may have been chemically synthesized in various embodiments and/or recombinantly produced using protein design. Nowadays, short peptides can easily be prepared synthetically, for example using solid-phase synthesis such as Merrifield's solid-phase synthesis. Longer peptides and polypeptides, on the other hand, are often produced recombinantly in the host organism, e.g. in bacteria or yeast.

It is preferred to produce the peptides and/or peptide conjugates according to the disclosure using recombinant processes. This includes all genetic engineering or microbiological processes that are based on the genes for the peptides of interest being introduced into a host organism suitable for production and transcribed and translated by it (summarized in the context of this disclosure as biotechnological processes).

The peptides and/or peptide conjugates according to the disclosure are particularly preferably produced as polypeptides (multimers) and subsequently cleaved into the functional peptides and/or peptide conjugates. Very particularly preferred multimers have 1 to 30 peptide units (each according to the disclosure), each of which is separated from one another by spacers of 1 to 10 amino acids long (e.g. 1, 2, 3 or 4 amino acids). Alternatively, the spacers can also be or include interfaces for specific proteases/peptidases, in particular endopeptidases, or can form such an interface together with parts of the peptide.

Using methods that are generally known today, such as chemical synthesis or the polymerase chain reaction (PCR) in conjunction with standard molecular biological and/or protein chemical methods, it is possible for a person skilled in the art to identify the corresponding nucleic acids and even complete genes using known DNA and/or amino acid sequences to produce. Such methods are, for example, from Sambrook, J., Fritsch, E. F. and Maniatis, T. 2001. Molecular cloning: a laboratory manual, 3rd Edition Cold Spring Laboratory Press. known.

In particularly preferred embodiments, peptides and/or peptide conjugates described herein are produced using biotechnological processes as described above. The at least one peptide and/or peptide conjugate according to the disclosure is preferably suitable for the inducement of an immune response against uveal melanoma cells.

### Abbreviations:

CBT immune checkpoint blockade therapy
Cy cyclophosamide
DLTs dose-limiting toxicities
GNA11 guanosine nucleotide-binding protein alpha-11
GNAQ guanosine nucleotide-binding protein Q
MP melanoma peptides
MHP melanoma helper peptide
RFS recurrence-free survival.
SNV single nucleotide variations
TMB tumor mutation burden
UM uveal melanoma

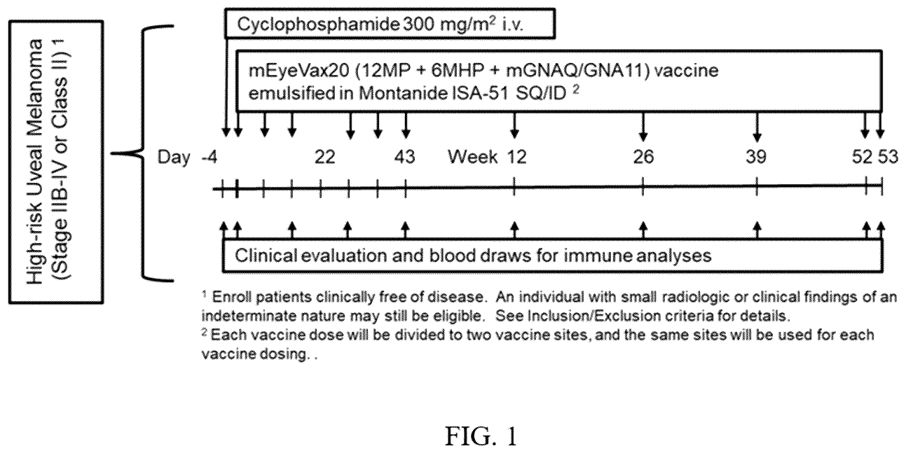

FIG. 1 presents a protocol schema for the administration of a UM vaccine and/or adjuvant treatment. As above discussed in brief, patients receiving embodiments of the invention may be deemed eligible for reception of inventive embodiments after passing certain eligibility requirements. In some embodiments, the patient may have high-risk UM as categorized following excision or other definitive therapy. In certain embodiments, UM risk level may be determined by considering: the outcome of the DecisionDX-UM protocol (i.e., so-called “Class II” cases); PRAME expression in a UM tumor; ECOG performance status 0-1; presence or absence of any major organ dysfunction; presence or absence of any active immunosuppression; or, pregnancy status. One or more elements alone or in combination may be used to determine suitability for administration of embodiments of the disclosed compositions.

Further seen in FIG. 1, in certain embodiments, eligible patients will receive 1 dose of Cy (300 mg/m2) day-4, then mEyeVax20 compositions emulsified in incomplete Freund's adjuvant (IFA, Montanide ISA-51; France). In certain embodiments, composition administration subsequently occurs on days 1, 8, and 15. Vaccine compositions may be administered (half-intradermally and half-subcutaneously in some embodiments) at 2 injection sites (primary and replicate sites), on extremities uninvolved with the UM tumor. On day 22, an optional biopsy may be performed of a lymph node draining the replicate vaccine site (sentinel immunized node, SIN). On days 29, 36, and 43, one vaccine may be administered at the primary vaccine site only. Patients may be reassessed at weeks 8 and 12 for evidence and confirmation of clinical response via Response Evaluation Criteria in Solid Tumor (RECIST) v1.0. Patients without progression may be eligible for 6 cycles of 3 weekly booster vaccines, until progression or for 2 years.

As above briefly discussed, embodiments of the compositions herein disclosed include mEye Vax20 which comprises twenty (20) total peptides (12MP+6MHP+GNAQ+GNA11). Table 1 presents the full amino acid sequences for normal GNAQ (SEQ ID NO 1) And GNA11 (SEQ ID NO 2). GNAQ and GNA11 are highly homologous but differ in some residues. Table 1 shows highlighted by italics several amino acids around the common site for mutation (residue 209). The normal amino acid at position 209 is Q (glutamine, Q209), and it is most mutated so that the Q209 is replaced by L (leucine), a mutation known as Q209L. The next most common mutation is where the Q209 is replaced by proline (P), and this is referred to as Q209P. The italicized sub-sequences represent 9 amino acids on either side of residue 209 (shown as the Q bolded, underlined, and without italics) The two sequences are 100% homologous between GNAQ and GNA1. Thus, two peptides represent three driver mutations.

TABLE 1
STANDARD SEQUENCES OF GNAQ AND GNA11

| Peptide | SEQ ID NO | Sequence |
|---|---|---|
| GNAQ | 1 | MTLESIMACCLSEEAKEARRINDEIERQLRRDKRD |
| | | ARRELKLLLLGTGESGKSTFIKQMRIIHGSGYSDE |
| | | DKRGFTKLVYQNIFTAMQAMIRAMDTLKIPYKYE |
| | | HNKAHAQLVREVDVEKVSAFENPYVDAIKSLWN |
| | | DPGIQECYDRRREYQLSDSTKYYLNDLDRVADPA |
| | | YLPTQQDVLRVRVPTTGIIEYPFDLQSVIFRMVDVG |
| | | GQRSERRKWIHCFENVTSIMFLVALSEYDQVLVES |
| | | DNENRMEESKALFRTIITYPWFQNSSVILFLNKKD |
| | | LLEEKIMYSHLVDYFPEYDGPQRDAQAAREFILK |
| | | MFVDLNPDSDKIIYSHFTCATDTENIRFVFAAVKD |
| | | TILQLNLKEYNLV |
| GNA11 | 2 | MTLESMMACCLSDEVKESKRINAEIEKQLRRDKR |
| | | DARRELKLLLLGTGESGKSTFIKQMRIIHGAGYSE |
| | | EDKRGFTKLVYQNIFTAMQAMIRAMETLKILYKY |
| | | EQNKANALLIREVDVEKVTTFEHQYVSAIKTLWE |
| | | DPGIQECYDRRREYQLSDSAKYYLTDVDRIATLG |
| | | YLPTQQDVLRVRVPTTGIIEYPFDLENIIFRMVDVG |
| | | GQRSERRKWIHCFENVTSIMFLVALSEYDQVLVES |
| | | DNENRMEESKALFRTIITYPWFQNSSVILFLNKKD |
| | | LLEDKILYSHLVDYFPEFDGPQRDAQAAREFILKM |
| | | FVDLNPDSDKIIYSHFTCATDTENIRFVFAAVKDTI |
| | | LOLNLKEYNLV |

Overall, 91-100% of uveal melanomas (UM) express melanocytic differentiation antigens gp100, tyrosinase, and MART-1/MelanA, and on 75-100% of cells in those tumors. Cancer-testis antigens (CTAs) included in the proposed vaccine are from MAGE-A1, A2, A3, NY-ESO-1. In one study, expression of MAGE-A1, MAGE-A3/A6, and NY-ESO-1 was reported in ˜5%, 25%, and 0% of UMs; however, other studies have reported that MAGE-A1, A2, A3 were highly expressed in the primary and metastases of a patient with UM and that MAGE-A1 and A3 were expressed in 41% and 53% of cell lines from 17 UMs. Mutations of GNAQ and GNA11 at the Q209 residue are detected in 41% and 45% of patients, with 45% having GNA11 Q209L, 31% GNAQ Q209P and 10% GNAQ Q209L (from TCGA, n=108).

Additional embodiments may include peptides from the PRAME protein (or variants thereof). PRAME is selectively expressed in melanomas and is highly expressed in uveal melanomas.

In certain embodiments, the vaccine compositions include peptide antigens presented by HLA-I and HLA-II to CD8+ and CD4+ T cells, respectively. The source proteins and example peptide sequences in the an exemplary vaccine composition may be found in Table 2.

TABLE 2
SOURCE PROTEINS AND EXAMPLE PEPTIDE SEQUENCES FOR AN
EXAMPLE EMBODIMENT.

| | | | | | 6MHP |
|---|---|---|---|---|---|
| | % of | | | | (promiscuous for |
| Source | UM | SEQ | 12MP (Class I MHC) | SEQ | Class II MHC) |
| Protein | Expressing | ID NO | NSC # 728925 | ID NO | NSC # 728926 |
| gp100 | 90- | 3 | IMDQVPFSV (A2) | 17 | WNRQLYPEWTEA |
| | 100% | 4 | YLEPGPVTA (A2) | | QRLD |
| | | 5 | ALLAVGATK (A3) | | |
| | | 6 | LIYRRRLMK (A3) | | |
| Tyrosinase | 90- | 7 | DAEKSDICTDEY | 18 | AQNILLSNAPLGP |
| | 100% | | (A1) | | QFP |
| | | 8 | SSDYVIPIGTY (A1) | 19 | FLLHHAFVDSIFE |
| | | 9 | YMDGTMSQV (A2) | | QWLQRHRP |
| MelanA/ | 90- | | — | 20 | RNGYRALMDKSL |
| MART-1 | 100% | | | | HVGTQCALTRR |
| MAGE-A1 | ~5% | 10 | EADPTGHSY (A1) | — | |
| | | 11 | SLFRAVITK (A3) | | |
| MAGE- | ~25% | | — | 21 | LLKYRAREPVTKA |
| A1, A2, A3, | | | | | E |
| A6 | | | | | |
| MAGE-A3 | ~25% | 12 | EVDPIGHLY (A1) | 22 | TSYVKVLHHMVK |
| | | | | | ISG |
| MAGE-A10 | Unk. | 13 | GLYDGMEHL (A2) | — | |
| NY-ESO-1 | 0% | 14 | ASGPGGGAPR (A3, | 23 | GARGPESRLLEFY |
| | | | A31, A33) | | LAMPFATPMEAEL |
| | | | | | ARRS |
| GNA11 Q209L | 45% | 15 | IFRMVDVGGLRSERRKWIH | | |
| GNAQ Q209L | 10% | | | | |

| | | | |
|---|---|---|---|
| GNAQ Q209P | 31% | 16 | IFRMVDVGGPRSERRKWIH |
| PRAME | 26-45% | 26 | SLLQHLIGL (A2) |
| | | 27 | ELFSYLIEK (A3) |
| | | 28 | RPRRWKLQVL (B7) |
| | | 29 | KVKRKKNVL (B8) |
| | | 30 | TSPRRLVELAGQSLLKDEA |
| | | | (Class II MHC) |
| | | 31 | DELFSYLIEKVKRKKNVLR |
| | | | (Class II MHC) |

Thus, in certain embodiments of the disclosure, the sequence for GNAQ is FDLQSVIFRMVDVGGORSERRKWIHCFENVT (SEQ ID NO 24) and in the same or different embodiments the sequence for GNA11 is FDLENIIFRMVDVGGORSERRKWIHCFENVT (SEQ ID NO 25). As noted above, GNAQ and GNA11 are highly homologous but are mismatched for 4 amino acids separated by 9 residues from the Q at 209 (bolded and underlined in above sequences with SEQ ID Nos. 24 and 25).

Thus, in certain embodiments of the disclosure, the sequence for GNAQ is: FDLQSVIFRMVDVGGORSERRKWIHCFENVT (SEQ ID NO 24) and in the same or different embodiments the sequence for GNA11 is FDLENIIFRMVDVGGORSERRKWIHCFENVT (SEQ ID NO 25). As above noted, GNAQ and GNA11 are highly homologous but are mismatched for 4 amino acids separated by 9 residues from the Q at 209 (bolded and underlined in above sequences with SEQ ID Nos. 24 and 25).

In certain other embodiments, the 12MP and 6MHP peptides may be presented in Tables 3 and 4.

TABLE 3
ADDITIONAL 12-MP PEPTIDES.

| Allele | SEQ ID NO | Sequence | Epitope |
|---|---|---|---|
| HLA-A1 | 7 | DAEKSDICTDEY | Tyrosinase₂₄₀₋₂₅₁* |
| | 8 | SSDYVIPIGTY | Tyrosinase₁₄₆₋₁₅₆ |
| | 10 | EADPTGHSY | MAGE-A1₁₆₁₋₁₆₉ |
| | 12 | EVDPIGHLY | MAGE-A3₁₆₈₋₁₇₆ |
| HLA-A2 | 9 | YMDGTMSQV | Tyrosinase₃₆₉₋₃₇₇^{♦} |
| | 3 | IMDQVPFSV | gp100₂₀₉₋₂₁₇^{#} |
| | 4 | YLEPGPVTA | gp100₂₈₀₋₂₈₈ |
| | 13 | GLYDGMEHL | MAGE-A10₂₅₄₋₂₆₂ |
| HLA-A3 | 5 | ALLAVGATK | gp100₁₇₋₂₅ |
| | 6 | LIYRRRLMK | gp100₆₁₄₋₆₂₂ |
| | 11 | SLFRAVITK | MAGE-A1₉₆₋₁₀₄ |
| | 14 | ASGPGGGAPR | NY-ESO-1₅₃₋₆₂ |

*(substitution of S for C at residue 244)
^{♦}(post-translational change of N to D at residue 371)
^{#}(209-2M, substitution of M for T at residue 210)

TABLE 4
ADDITIONAL 6-MHP PEPTIDES.

| Allele | SEQ ID NO | Sequence | Epitope |
|---|---|---|---|
| HLA-DR1 | 17 | WNRQLYPEWTEAQRLD | gp100₄₄₋₅₉ |
| & -DR4 | | | |
| HLA-DR4 | 18 | AQNILLSNAPLGPQFP | Tyrosinase₅₆₋₇₀^{#} |
| HLA- | 19 | FLLHHAFVDSIFEQWLQRHRP | Tyrosinase₃₈₆₋₄₀₆ |
| DR15 | | | |
| HLA-DR4 | 20 | RNGYRALMDKSLHVGTQCALTRR | Melan-A/MART-1₅₁₋₇₃ |
| HLA- | 21 | LLKYRAREPVTKAE | MAGE-1,2,3,6₁₂₁₋₁₃₄ |
| DR13 | | | |
| HLA- | 22 | TSYVKVLHHMVKISG | MAGE-3₂₈₁₋₂₉₅ |
| DR11 | | | |

^{#}An alanine residue was added to the N-terminus to prevent cyclization.

In other embodiments, the GNA11 and GNAQ peptides may be those as found in Table 5.

TABLE 5
ALTERNATIVE SEQUENCES OF GNAQ AND GNA11 PEPTIDES FOR
VACCINE USAGE

| Peptide | SEQ ID NO | Sequence |
|---|---|---|
| GNA11 Q209L | 32 | YPFDLENIIFRMVDVGGLRSERRKWIHCFEN |
| GNAQ Q209L | 33 | YPFDLQSVIFRMVDVGGLRSERRKWIHCFEN |
| GNAQ Q209P | 34 | YPFDLQSVIFRMVDVGGPRSERRKWIHCFEN |

In additional embodiments the peptides of Table 5 may be shortened to remove the “YP” at the N terminus for MHC II binding. The peptides may only need be as long as the “WI” residues at the C terminus for MHC I binding. Examples are presented in Table 6.

TABLE 6
SHORTENED SEQUENCES OF GNAQ AND GNA11 PEPTIDES FROM TABLE
5 (FOR MHC II BINDING).

| Peptide | SEQ ID NO | Sequence |
|---|---|---|
| GNA11 Q209L | 35 | FDLENIIFRMVDVGGLRSERRKWIH |
| GNAQ Q209L | 36 | FDLQSVIFRMVDVGGLRSERRKWIH |
| GNAQ Q209P | 37 | FDLQSVIFRMVDVGGPRSERRKWIH |

In additional embodiments, the peptides may be further shortened for the targeting/activation of CD8 T cells (for Class I MHC binding) as shown in Table 7.

TABLE 7
SHORTENED SEQUENCES OF GNAQ AND GNA11 PEPTIDES FOR CD8 T
CELLS (FOR CLASS I MHC BINDING).

| MHC | SEQ ID NO | Sequence |
|---|---|---|
| HLA-A*03:01 | 38 | RMVDVGGLR |
| HLA-B*27:05 | 39 | FRMVDVGGLR |
| HLA-A*03:01 | 40 | RMVDVGGPR |
| HLA-B*07:02 | 41 | GPRSERRKW |
| HLA-B*44:02 | | |

The examples provided in Tables 6-7 represent sequences for MHC (major histocompatibility complex) binding as part of the Human Leukocyte Antigen (HLA) system. Those of skill in the art can readily ascertain peptides that may be characterized as “strong binders” for the peptide-binding grooves of MHC class 1 and class II molecules for interactions with CD8+ (cytotoxic T cells) and CD4+ (helper T Cells). It is these interactions that trigger the overall immune system for purposes of prevention, elimination, or reduction of UM.

### Kits/Article of Manufacture

Disclosed herein, in certain embodiments, are kits and articles of manufacture for use to generate or dose embodiments of the disclosed vaccines in accordance with one or more methods described herein. In some embodiments, described herein is a kit for the administration of a multi-valent vaccine to a patient. In some embodiments, such kit includes buffers, stabilizers, media and other solutions as needed to stabilize peptides prepared in accord with embodiments of the disclosure.

In some instances, the kit further comprises suitable solutions such as buffers or media for resuspension and administration of peptides in a pelleted form. In some embodiments, the kit further comprises purified or substantially purified peptides for use in one or more of the methods described herein. In some embodiments, additional components of the kit comprise a carrier, package, or container that is compartmentalized to receive one or more containers such as vials, tubes, and the like, each of the container(s) comprising one of the separate elements to be used in a method described herein. Suitable containers include, for example, bottles, vials, plates, syringes, and test tubes. In one embodiment, the containers are formed from a variety of materials such as glass or plastic.

The articles of manufacture provided herein contain packaging materials. Examples of pharmaceutical packaging materials include, but are not limited to, bottles, tubes, bags, containers, and any packaging material suitable for a selected formulation and intended mode of use.

For example, the container(s) include probes, test compounds, and one or more reagents for use in a method disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein without any additional undue experimentation. It being understood that various changes may be made in the function and arrangement of elements described in an exemplary embodiment without departing from the scope of the various embodiments as set forth in the appended claims.

Since certain changes may be made in the above-described disclosure, without departing from the spirit and scope of the disclosure herein involved, it is intended that all of the subject matter of the above description shown in the accompanying drawings shall be interpreted merely as examples illustrating the inventive concept herein and shall not be construed as limiting the disclosure.

Finally, the written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A multi-peptide composition, comprising:
(a) at least one peptide selected from each of the following source proteins: gp100, tyrosinase, MelanA/MART-1, MAGE-A1, MAGE-A3, MAGE-A10, and NY-ESO-1, wherein said peptides are selected for binding to Class I major histocompatibility complex (MHC) molecules;
(b) at least one peptide selected from each of the following source proteins: gp100, tyrosinase, MelanA/MART-1, MAGE-A1, MAGE-A3, and MAGE-A6, wherein said peptides are selected for binding to Class II major histocompatibility complex (MHC) molecules;
(c) at least one peptide comprising a mutated sequence from GNAQ or GNA11, wherein the mutation is at residue 209 and is selected from Q209L or Q209P.

2. The composition of claim 1, wherein the composition comprises twenty peptides, including:
twelve Class I MHC-binding peptides;
six Class II MHC-binding peptides; and
two peptides comprising the Q209L and Q209P mutations.

3. The composition of claim 2, wherein the twelve Class I MHC-binding peptides are selected from the group consisting of: IMDQVPFSV (SEQ ID NO 3), YLEPGPVTA (SEQ ID NO 4), ALLAVGATK (SEQ ID NO 5), LIYRRRLMK (SEQ ID NO 6), DAEKSDICTDEY (SEQ ID NO 7), SSDYVIPIGTY (SEQ ID NO 8), YMDGTMSQV (SEQ ID NO 9), EADPTGHSY (SEQ ID NO 19), EVDPIGHLY (SEQ ID NO 12), GLYDGMEHL (SEQ ID NO 13), SLFRAVITK (SEQ ID NO 11), and ASGPGGGAPR (SEQ ID NO 14).

4. The composition of claim 2, wherein the six Class II MHC-binding peptides are selected from the group consisting of AQNILLSNAPLGPQFP (SEQ ID NO 18), FLLHHAFVDSIFEQWLQRHRP (SEQ ID NO 19), RNGYRALMDKSLHVGTQCALTRR (SEQ ID NO 20), TSYVKVLHHMVKISG (SEQ ID NO 22), LLKYRAREPVTKAE (SEQ ID NO 21), and WNRQLYPEWTEAQRLD (SEQ ID NO 17).

5. The composition of claim 2, wherein the peptides comprising a mutated sequence are:
the Q209L peptide
(SEQ ID NO 15)
IFRMVDVGGLRSERRKWIH;
and,
the Q209P peptide
(SEQ ID NO 16)
FRMVDVGGPRSERRKWIH.

6. The composition of claim 1, wherein the pharmaceutically acceptable preparation is formulated as an oil-in-water emulsion comprising incomplete Freund's adjuvant.

7. The composition of claim 1, wherein the composition is formulated for intradermal or subcutaneous administration.

8. The composition of claim 1, wherein each peptide is synthesized under GMP conditions and provided in lyophilized single-use vials.

9. The composition of claim 1, further comprising a pharmaceutically acceptable carrier selected from water, saline, Ringer's solution, dextrose solution, and human albumin.

10. The composition of claim 1, further comprising:
a kit for administration, wherein the kit includes:
(a) one or more containers comprising the multi-peptide composition;
(b) a pharmaceutically acceptable buffer or media for resuspension of the peptides;
(c) optionally, instructions for use of the composition in the treatment of uveal melanoma; and,
(d) packaging materials suitable for storage and transport of the composition.

11. A method, comprising:
administering to a human the composition of claim 1.

12. The method of claim 11, wherein the composition of claim 1 is administered half intradermally and half subcutaneously at two injection sites on days 1, 8, and 15 following pre-treatment with cyclophosphamide at a dosage of about 300 mg/m2 administered four days before the first peptide administration.

13. The method of claim 11, further comprising administering booster doses of the composition of claim 1 at three-week intervals for up to two years in the absence of disease progression.

14. The composition of claim 1, wherein the peptides comprising a mutated sequence from GNAQ or GNA11 further include one or more peptides selected from the group consisting of RMVDVGGLR (SEQ ID NO 38), FRMVDVGGLR (SEQ ID NO 39), RMVDVGGPR (SEQ ID NO 40), and GPRSERRKW (SEQ ID NO 41).

15. A multi-peptide composition, comprising:
(a) sixteen peptides selected for binding to Class I major histocompatibility complex (MHC) molecules, each peptide derived from a source protein selected from the group consisting of gp100, tyrosinase, MelanA/MART-1, MAGE-A1, MAGE-A3, MAGE-A10, NY-ESO-1, and PRAME;
(b) eight peptides selected for binding to Class II major histocompatibility complex (MHC) molecules, each peptide derived from a source protein selected from the group consisting of gp100, tyrosinase, MelanA/MART-1, MAGE-A1, MAGE-A3, MAGE-A6, and PRAME;
(c) two peptides comprising mutated sequences from GNAQ or GNA11, wherein the mutation is at residue 209 and is selected from Q209L or Q209P;
wherein the composition consists of twenty-six peptides in total and is formulated as a pharmaceutically acceptable preparation.
